(19) United States
(12) Patent Application Publication
(10) Pub. No.: US 2005/0142224 A1
(43) Pub. Date: Jun. 30, 2005

(54) Pharmaceutical activity and manufacturing method of the four plants compound wares

(76) Inventors: Jiao Gong, Arcadia, CA (US); Wei Jiang, Arcadia, CA (US)

Correspondence Address: James Chen, 150 N Santa Anita Ave., #560, Arcadia, CA, 91006, US

(21) Appl. No.: 10/746,363

(22) Filed: Dec. 29, 2003

(51) Int. Cl. A61K 35/78

(52) U.S. Cl. 424/728; 424/757; 424/773

(57) ABSTRACT

Clearing the free radicals in the body and suppressing the specific marker MAO-B's activity are the special dedication to human being by American scientists. Defering senility and raising life quality are the original pursue of the human being, and looking for a drug for deferring senility is the American dream for long. The four plant compound wares use the WLD resin for absorption, elution and separation and purifying the plant component under the careful and strict condition. Quatity measure with high efficiency liquid phase chromatography meter ensure the equal and stable product. The pharmacological experiment show that the four plant compound wares have the pharmacological activities of raising SOD activity in the old moice's livers, increasing SOD content in the blood notably, supressing MAO-B's vitality significantly, adding up the catalase vitality in the erythrocyte. It gets to the unimagble effect. The extensive use of the compound wares in the medical domain is according to its pharmacological activity.

### BACKGROUND OF THE INVENTION

The pharmacology activity and making method about the four plant compound wares' postpone aging are combination patent in the United State. It separate and purify the natural products. The four plant compound wares can significant reinforce the activity of SOD of advanced aged mice, raise the level of SOD in the mice's blood, suppress the activity of monoamine oxidase B(MAOB) in the rat liver mitochondria, and strenth the catalase's activity in the mice's erythocyte.

If an invention is claimed to treat human or animal's disease and determine whether the utility is qualified to the demand of the patent legislation, the standard is the same as the others stipulated by laws in the other domais. In the patent legislation, we emphasize in particular that the utility must be separated from FDA about the safety and efficacy in America. The base of evaluating the sort of the invention's utility is the activity of the invention's treatment, prevention and pharmacology. It is to confirm the invention's pharmacological activity and get to the demand of utility.

The world is getting into the aged times, and the life span is longer and longer. Excogitating and looking for the anti-senile measures and drugs has great significance to senile biology and gerontology and to raise the quality of human's life. Postponing senility drugs have the effect of deferring the ageing, elongating the life span and elevating the quality of life. The modes are Superoxide Dismutase SOD and monoamine oxidase inhibitor (MAOI) in the United States. SOD is a protein contained Zinc, iron, Manganese, and it is against a lot of oxygen-derived free radidicals. It is an important anti-oxidase, and it can clear the free radidicals. Too much puperoxide anion free radicals can cause Lipid peroxidation, then cellular damage, inflammation, tumor and autoimmunity disease. It can also accelerate the body's ageing, and SOD can clear the free radicals, can prevention and cure some senium disease and defer the senility. SOD is derived from the erythrocyte, liver cell of the animals, which be separated and laid in the aqueous solution in American hospital copper, Znic and SOD is exogenous Producing and processing are more complicated. Taking orallyis easy to be destructed by gastric The acid monoamine oxidase(MAO) takes part in monoamine substance metabolism, the activity of MAO is higher and higher by the age. MAO's rising may be the important factor of the monoamine substance's decrease in the old's brains. Deficiency of monoamine transmitter in the brain has relation to senility. Using MAO inhibitor can increase monoamine transmitters in the brain, and prevent some physiological function diminution, then defer the senility in particular the brains.

### BRIEF SUMMARY OF THE INVENTION

Postponing the senility and improving the life quality are the original pursue of the human being. Looking for the drugs for deferring senility is the human being's dream for long. The America is coming into an aged community structure. The aged people and their problems are one kind of most important problems little by little.

The body's senescence is the cell's, and the nature of the cell's senescence is that of cellular function. The free radicals can result the irreversible damage. MAO is the mark of senescence in human being's brain. The pharmacology experiment about the four plant compound wares ‘defering senescence showed that the compound wares could raise the activity of SOD in the body, and it could prevent the compound wares from the free radicals' oxidizing damage. In the experiment, the activity of SOD in young mouse is higher than that in the old. It suggested that the activity of SOD decreased when the age was old. The compound wares can significantly strength the activity of SOD in the aged mouse’ livers and bloods. MAO-B is one of the enzymes related to brain's senescence. As the mouse grew, MAO-B's activity is higher. In vitro the compound suppress MAO-B depending on dosage. We observed the compound wares activation to catalase, then knew its antioxidation. The experiment showed that the compound wares could add vigor of anticatalase in the mouse erythrocyte and raise the body's ability of anti Oxidation and anti senescence.

### BRIEF DESCRIPTION OF THE SEVERAL VIEWS OF THE DRAWING

TABLE 1
the material resource and conformation of the four plant compound wares

| | | |
|---|---|---|
| 1. | Radix Angelicae Sinensis | 28% |
| | Umbelliferae | |
| | Angelica Sinensis(Dliv.)Diels's root | |
| 2. | Radix Polygoni Multiflori | 22% |
| | Polygonaceae | |
| | Polygonum Multiflorum Thunb's root | |
| 3. | Radix Astragali | 28% |
| | Leguminosae | |
| | Astragalus Menbranaceus(Fisch.) | |
| | Bge.Var.Mongholicus(Bge.)Hsiao's root | |
| 4. | Radix Ginseng | 22% |
| | Araliaceae | |
| | Panax Ginseng C.A.Mey.'S root | |
| | Total | 100% |

TABLE 2
The making method and craftwork processing of the four plant compound wares

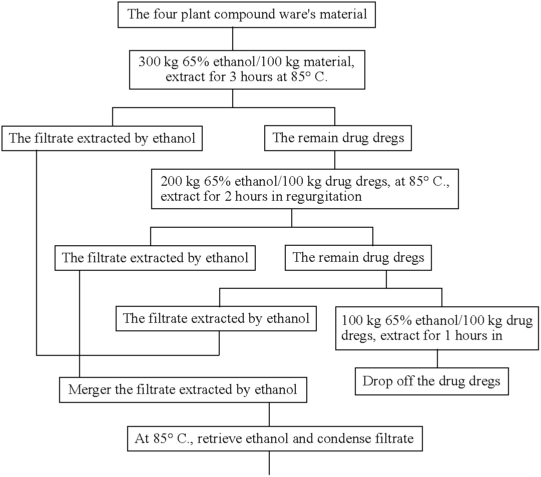

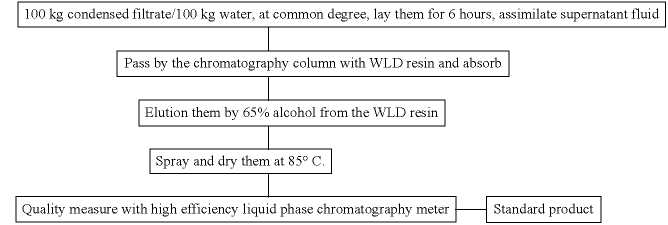

Table 3 the Four Plant Postponing Senescence Wares' Pharmacological Activity

1. The four plant postponing senescence wares (for short: compound wares) have effects on the activity of superoxide dismutase (SOD) in mouse' livers.

The pharmacology experiment about the four plant compound wares' deferring senescence showed that the compound wares could raise the activity of SOD in the body, and it could prevent the compound wares from the free radicals' oxidizing damage.

(1) the young control group, the old control group, given compound wares old rat group, every group has ten. Every rat takes 50 mg wares every day for 60 days, and the control group takes Saline.

(2) enzyme fluid established: take proper mount fresh liver, pick out connective tissue, wash out the blood dirty, add five times 0.1 mmol/L EDTA-0.05 mol/L phosphoric acid buffer (ph 7.8), use glass homogenizer ice bath milling to 20% homogenate. Take 1.5 ml homogenate, put it into the centrifuge tube, 15000 rpm centrifugation for 9 minutes, suck supernatant fluid and transfer to another tube, lay it at common degree for a time.

(3) Enzyme' activity assay: take enzyme fluid 0.05 ml, put into exponent tube, add diplo-distilled water 0.05 ml, add diplo-distilled water 0.1 ml into vacuity tube, add Met-NBT fluid 3.8 ml, add hepatoflavin fluid 0.1 ml in the end, after mix by turbulence blender sufficiently, add up tampon, lay them at 20 W under the daylight lamp about 40 cm for twenty minutes, illumination is 4000/oux, measure OD value at 560 nm at once by chromatometry. Establish a vacuity tube in the same way for adjusting zero. Measure protein mount in the enzyme fluid by Lowry, the standard as calfalbumin. Operation sequence is as the following:

| | Vacuity tube | Assay tube |
|---|---|---|
| Enzyme fluid | — | 0.05 ml |
| Diplo-distilled water | 0.1 ml | 0.05 ml |
| Met-NBT | 3.8 ml | 3.8 ml |
| Hepatoflavin fluid | 0.1 ml | 0.1 ml |

Mix by turbulence blender sufficiently, add up tampon, lay them at 20 W under the daylight lamp about 40 cm for twenty minutes, illumination is 4000/ou, measure OD value at 560 nm. According to the OD value, calculationsod enzyme specific activity on the following formula:

$$\text{Antinbt recovery percentage (\%)} = ( Vacuity tube OD - Assay tube OD ) / Vacuity tube OD \times 100 \%$$

$$50 µl exponent SOD enzyme activity = \frac{\text{antinbt recovery percentage}}{50 \%}$$

$$SOD activity Unit / ml = 50 µl exponent SOD activity Unit \times 20$$

$$SOD enzyme specific activity ( u \text{/} mg \cdot h ) = \frac{SOD activity unit ( u \text{/} ml )}{protein content ( mg \text{/} ml )} \times 3$$

Result: In the experiment, the activity of SOD in young mouse is higher than that in the old. It suggested that the activity of SOD decreased when the age was old. The compound wares can significantly strength the activity of SOD in the aged mouse's livers. (table 1).

TABLE 1
the compound wares have effects on the activity of SOD in the
mouse's liver(n = 10)

| | SOD activity | SOD relative |
|---|---|---|
| Group | (u/mg · h) X ± SD | activity (%) |
| Young murine control | 2.14 ± 0.23 | 227.66 ± 24.47*** |
| Old murine control | 0.94 ± 0.06 | 100.00 ± 6.38 |
| Given compundwares | 1.50 ± 0.18 | 159.57 ± 19.15** |
| old murine | | |

2. The Compound Wares have Effects on the Content of SOD in the Mouse's Blood

Eighteen mouse are divided into groups at random. The experimental groups perfus 50, 100 mg/kg compound wares respectively, the same volume in the control group. After doing that for continous fifteen days, at the 16^{th} day get 20 μl blood by shearsing the tails, add 40 μl distilled water, shake them equal for making hemolysis. Measure ferrohemoglobin by Hemoglobin determination meter by colorimetric assay by eyes. Put 10 μl hemolysis fluid into color immune board foramen. Then take the board into the box to keep wet (ordinary lunch box with carbasus), lay them at common degree for over than 15 hours. The color immune board is with measurement ruler. About the response foramen, we can see the fine and clear ring, which color is heavier than the backgroud. That is the Antigen-antibody complex pigmented. Measure the diameter of the deposit ring. Look up the SOD content in the standard curve, calculate the SOD content according the following formula:

$$SOD µg / ghb = \frac{10 µl hemolysis fluid SOD µg}{3.33 µl entirety gram}$$

In the formula 3.33 is problood in the 10 μl hemolysis fluid.

Result: the compound wares can add sodcontent in the murine's blood significantly (table2).

TABLE 2
the compound wares' effect on SOD content in the murine's blood

| | | | SOD content |
|---|---|---|---|
| Group | Dosage | Number (n) | (μg/gHB, X ± SD) |
| Saline | — | 6 | 488.0 ± 65.3 |
| The compound wares | 50 mg/kg | 6 | 614.2 ± 59.6*** |
| The compound wares | 100 mg/kg | 6 | 625.2 ± 63.1*** |

3. The Four Compound Wares' Effect s on Monoamine Oxidase B(MAOB) in the Rat Liver Mitochondrion

The four plant compound wares have effects on suppressing senility. MAO-B is one of the enzymes relatived to anti-senescence. MAO-B's activity is stronger as the rat grows. We study the mechanism by invesgating the compound wares' effects on MAO-B.

(1) Two rat weighing 250 g, killed by luxation, select the liver quickly, remove the blood dirty and weigh it. Add up 12 times (W/V) 0.25 mol/L saccharu solutions, establish the homogenate by constitution homogenate machine. Every 700 g centrifugate for ten minutes, remove the cell fragment. Supernatant fluid at 4° C., 1500 g centrifugating for 10 minutes. Take deposits into the saccharu solutions, centrifugate again at 15000 g for 10 minutes. The deposits are the mitochondrion involved MAO. Add up four timesas the liver wighted saccharu solutions, remix them and storage at −20° C.

(2) MAO-B activity measure: response architecture 0.3 ml, 0.3 mol/Lphosphoric acid buffer, 0.4 ml, 8 mmol/L benzyaminel, 0.7 ml enzyme fluid, 0.1˜0.6 ml compound wares, adjust the end volumn to 2 ml by distilled water. Add enzyme to start the response. At 37° C., agitation thermo-fostering for an hour, add up 30% trichloroacetic acid 0.2 ml to stop the response. The response product phenyl aldehyde cyclohexamide 4 ml are extracted, then centrifugate at 3000 rpm for 10 minutes, measure the supernatant fluid at 242 nm. The procedure is as the following:

In vitro MAO-B activity measure procedure

| | Vacuity | Contrast | Exponent |
|---|---|---|---|
| | tube | tube | tube |
| 0.3 mol/L phosphoric acid Buffer (ml) | 0.3 | 0.3 | 0.3 |
| 8 mol/L benzyaminel (ml) | 0.4 | 0.4 | 0.4 |
| Compound wares (ml) | 0 | 0 | 0.1˜0.6 |
| Distilled water (ml) | 0.6 | 0.6 | 0˜0.5 |
| Enzyme fluid (ml) | 0 | 0.7 | 0.7 |
| 30% trichloroacetic acid (ml) | 0.2 | 0 | 0 |

37° C. agitation thermo-fostering for 60 min

| | | | |
|---|---|---|---|
| 30% trichloroacetic acid (ml) | 0 | 0.2 | 0.2 |
| Enzyme fluid (ml) | 0.7 | 0 | 0 |
| Circle Hexane (ml) | 4 | 4 | 4 |

After agitation, at 3000 rpm centrifugation for 10 min,
measure the supernatant fluid at 242 nm

Only distilled water but not compound wares' group: the vitality is 100%. The vacuity is added up trichloroacetic acid first, then enzyme fluid.

Result: in vitro the compound wares suppress the MAO-B vitality depending on the dosages (table 3).

TABLE 3
in vitro the compound wares' effects on the mitochondrions MAO-B's
activity in the rat liver

| | The wares' Concentration (mg/ml) | | | | | |
|---|---|---|---|---|---|---|
| | 50 | 100 | 150 | 200 | 250 | 300 |
| MAO-B suppress rate (%) | 10.8 | 17.5 | 30.1 | 37.9 | 53.9 | 64.1 |

4. The Four Plant Compound Wares' Effects on the Catalase Vitality in the Murine's Erythrocyte

The compound wares have anti-oxidation effect. We can know the mechanism of anti-oxidation by observating the compound wares' activation to the catalase.

(1) Male mice, weighing 18˜22 g, divided into two groups. In the given drugs group the compound wares are 100 mg/kg perfused to the stomach, the same volume saline in the contrast group. Once a day for fifiteen days. Drug withdrawal two days later, pick up the mouse's eyes for blood, with hepalean anticoagulant. Separate erythrocytes after centrifugation. Wash three times by saline, put the same volumn distilled water as expressed erythrocytes to the tube to be 1:1 hemolysis fluid.

(2) Catalase activity measure: take 30% H₂O₂ 0.5˜0.6 ml, add up water to 50 ml, take out 4 ml from that, add up ph7.0, 0.05 mol/L phosphoric acid buffer 26 ml, measure absorption value at 230 nm. If OD value is 0.5˜0.55, it can be the substrate fluid for catalase. Take 25° C. [symbol] substrate solutions 3 ml, add 1:100 hemolysis fluid liquefied gas 0.01 ml at 25° C., measure OD value at 230 nm at once, once a minute, caculate the catalase vitality according to the following formula:

$$K = \frac{2.303}{60} \cdot lg \frac{{OD}_{1}}{{OD}_{2}}$$

In the fomula CD₁ is OD value at zero at 230 nm, CD₂ is OD1 value at 230 nm on the first minute, catalase vitality=K/ghb.

Result: the compound wares can have notable effects on the catalase vitality in the murine's erythrocyte (table 4).

TABLE 1
the compound wares' effects on the catalase activity in the murine's
erythrocyte (x ± SD)

| | Animal number | Catalase vitality |
|---|---|---|
| Group | (n) | (K/ghb) |
| Saline | 10 | 37.8 ± 6.4 |
| Compound wares 100 mg/kg | 10 | 60.7 ± 13.3** |

### DETAILED DESCRIPTION OF THE INVENTION

1. The pharmacology activity and making method about the four plant compound wares' postpone aging are combination patent in the United State. The compound wares are Radix Angelicae Sinensis, Radix Polygoni multiflori, Radix Astragali, Radix Ginseng.

2. The four plant compound wares' material every 100 kg add up 300 kg, 200 kg, 100 kg water respectively, extract for 3, 2, 1 hours at 85° C. by ethanol, merger The filtrate extracted by ethanol Retrieve ethanol and concintration, 100 kg condensed filtrate/100 kg water, lay them for 6 hours and assimilate supernatant fluid. Pass by the chromatography column with WLD resin and absorb them, elute them by 65% alcohol from the WLD resin, spray and dry them at 85° C., quatity measure with high efficiency liquid phase chromatography meter, then standard product done.

43. The experiment about the mouse liver SOD activity show that the compound wares can significantly strength the activity of SOD in the aged mouse's livers. The experiment about the mouse blood SOD content show it can add up the SOD content in the mouse's blood very notable. About MAOB, it shows that the wares can significantly suppress the MAO-B's activity dependent on dosages. About the catalase activity it show that it can raise the catalase's vitality. The compound wares are excellent postponing senility drug by previous experiments.

1. I claim that Radix Angelicae Sinensis 28%, Radix Polygoni Multiflori 22%, Radix Astragali 28%, Radix Ginseng 22%, the four plant compound wares' pharmacological activity and making method are my invention:
I think my invention include the four plants, extracting by ethanol, adsorptionby WLD resin, elution, separation, purification and drying, measurement content and then standard product done.
I think in my invention the four plant compound wares have the pharmacological activities of raising SOD activity in the old moice's livers, increasing SOD content in the blood notably, supressing MAO-B's vitality significantly, adding up the catalase vitality in the erythrocyte.
